(19) United States
(12) Patent Application Publication
(10) Pub. No.: US 2022/0257799 A1
(43) Pub. Date: Aug. 18, 2022

(54) A RECEPTOR-TARGETING CONJUGATE WITH AN EFFECTIVE PHARMACOKINETIC PROFILE

(71) Applicant: FLUOGUIDE A/S, KØBENHAVN N (DK)

(72) Inventors: Andreas KJAER, FREDERIKSBERG (DK); Morten ALBRECHTSEN, CHARLOTTENLUND (DK)

(21) Appl. No.: 17/626,264

(22) PCT Filed: Jul. 15, 2020
(86) PCT No.: PCT/EP2020/069991

§ 371 Date: Jan. 11, 2022

(30) Foreign Application Priority Data
Jul. 16, 2019 (SE) 1950899-3

(51) Int. Cl. A61K 49/00; A61K 47/42

(52) U.S. Cl. CPC A61K 49/0034; A61K 49/0056; A61K 47/42

(57) ABSTRACT

The present invention describes a receptor-targeting conjugate comprising a fluorophore; a molecule, e.g. a peptide, binding to the receptor; and —a linker group which covalently links the fluorophore to the molecule binding to the receptor, wherein the conjugate is adapted to be administered intravenously into a human or animal body, and provide an effective pharmacokinetic profile with reference to inter alia receptor binding affinity and removal from plasma.

### FIELD OF THE INVENTION

The present invention relates to a receptor-targeting conjugate with a high receptor binding affinity in combination with an optimal pharmacokinetic profile intended for administration in a human or animal body.

### TECHNICAL BACKGROUND

There are known receptor-targeting conjugates intended for administration in a human or animal body. For instance, in WO 2016/041558 there is provided a conjugate composition that binds to the cell surface receptor uPA (uPAR). The conjugate is based on a fluorescence-labelled peptide useful as a diagnostic probe to the surfaces of cells expressing uPAR. The conjugate is capable of carrying a suitable detectable and imageable label that will allow qualitative detection of uPAR in vitro and in vivo. This renders the surgical resection of tumors more optimal.

One aim of the present invention is to provide an improved receptor-targeting conjugate for administration before imaging applications and/or surgery in cancer therapy, where the receptor-targeting conjugate has a combination of features including a high receptor binding affinity and an optimal pharmacokinetic profile for this type of use.

### SUMMARY OF THE INVENTION

The stated purpose above is achieved by a receptor-targeting conjugate comprising:
a fluorophore;
a molecule binding to the receptor; and
a linker group which covalently links the fluorophore to the molecule binding to the receptor,
   wherein the conjugate is adapted to
be administered systemically into a human or animal body;
provide receptor binding at least within 4,500 minutes, preferably 1,200 minutes, more preferably 600 minutes, even more preferably 300 minutes;
be removed from plasma to make the conjugate bound to the receptor visible, measured as plasma half-life which is the time it takes for the concentration in plasma to be reduced with 50%, and wherein the plasma half-life has a maximum of 4,500 minutes, preferably 1,200 minutes, more preferably 600 minutes, even more preferably 300 minutes;
provide receptor binding lasting at least 30 minutes;
   and wherein the receptor binding affinity, the time it takes to reach the desired receptor binding, the lasting of the receptor binding and plasma half-life translate into a TBR (tumor-to-background ratio) of at least 1.5 and reaches that level within 4,500 minutes, preferably 1,200 minutes, more preferably 600 minutes, even more preferably 300 minutes, after administration into the human or animal body and stays above 1.5 at least 30 minutes after that this level has been obtained.

In relation to the above it may be mentioned that the conjugate shown in WO 2016/041558 is not directed to providing an optimal pharmacokinetic profile where the receptor binding is held on a certain level, where such level is obtained in a short time after administration and where the removal from plasma is made such as according to the present invention with a short plasma half-life. TBR values for different alternatives are mentioned in WO 2016/041558, however both within and outside the level as provided for the receptor-targeting conjugate according to the present invention and inside and outside the speed the TBR is achieved and the time it lasts. And producing such values at a higher dose than the current invention. Nevertheless, and to summarize, the receptor-targeting conjugate according to the present invention exhibits a novel and optimal pharmacokinetic profile for certain receptors, certain administration types and indications.

### SPECIFIC EMBODIMENTS OF THE INVENTION

Below some specific embodiments of the present invention are presented and discussed further.

With reference to some expressions it may be mentioned that “blood” and “plasma” is sometimes used synonymously, however strictly speaking plasma is the yellowish liquid component of blood that normally holds the blood cells in whole blood in suspension. Plasma is the liquid part of the blood that carries cells and proteins throughout the body.

According to one specific embodiment of the present invention, the conjugate is a peptide conjugate and wherein the peptide conjugate comprises a peptide binding to the receptor.

Furthermore, the target receptor may be of different types according to the present invention. According to one specific invention, the targeting receptor is urokinase Plasminogen Activator Receptor (uPAR), tissue factor (TF), epidermal growth factor receptor (EGFR), prostate-specific membrane antigen (PSMA), Vascular Endothelial Growth Factor (VEGF), Folate receptor, matrix metalloproteinase-2 (MMP-2), membrane type-I MMP, transmembrane inhibitor of metalloproteinase-2 (TIMP2), CIC-3 chloride ion channels, disaccharides and other glycans or glyco-phosphatidylinositol (GPI)-anchored cell membrane receptors. Furthermore, the receptor types may have a proteolytic activity or other enzymatic activity bound or unbound to the cell surface, such as e.g. urokinase (uPA). Furthermore, the receptor is such expressed in human cancer, e.g. such correlated with a poor prognosis, local invasiveness, or metastasis. In relation to this it may further be mentioned that the conjugate product according to the present invention is predominantly/partly anchored to the outside of the cells expressing the specific receptor, i.e. in contrast to the conjugate product being internalized into the cells expressing the receptor.

In relation to the receptor it may also be mentioned that when saying that the receptor (uPAR) is expressed on cancer cells this may also imply that they are expressed on the bodies ‘normal’ stroma cells influenced by cancer cells they are in contact with or in extremely close proximity to (e.g. 2-5 cells in between). This is also true for cases where the ‘normal’ cells help the cancer cells in invading normal tissue. For clarity ‘normal’ stroma cells in such close proximity is also included as cancer cells. Normal in quotes as it can be argued that the cells under influence by cancer cells and expressing uPAR may no longer be termed normal.

As mentioned above, the receptor-targeting conjugate has an optimal receptor binding affinity and pharmacokinetic profile. The conjugate is injected systemically. Following administration of the conjugate, the conjugate will enter the bloodstream directly and immediately. This is followed by distribution of the conjugates through the circulatory system to all the tissues in the body. For tissue with blood flow (almost all tissue), containing the conjugate the entire tissue will light up as it is filled with blood containing the conjugated product. When such the tissue is exposed to light of the wavelength (color) being absorbed by the fluorophore contained in the conjugated product, it will re-emit light specific for the fluorophore contained in the conjugated product. The conjugate product binds to the receptors depending on its concentration, speed of binding and time it takes to fall off the receptor (unbind) which can be shown as:

R+L [symbol] RL

The reaction is characterized by the on-rate constant Kₒₙ and the off-rate constant K_{off}, which have units of 1/(concentration×time) and 1/time, respectively. The ration between the Kₒₙ and the K_{off} is the dissociation constant and a description of the receptor binding affinity.

The product conjugate will distribute into the blood and starts being removed from the blood, such as by being metabolized, excreted by the liver, eliminated by the kidney, or redistributes to distribution compartments other than the blood. When the difference between the conjugate product bound to the receptor and the unbound conjugate in proximity to the receptor, e.g. product circulating in the blood between the receptor bound conjugate and the light detection device, starts to increase, a relative difference in light identity is created between the receptor bound conjugate product and the unbound conjugate product. It results in the tissue with the presence of cells with the targeted receptor to where the conjugate product is bounds will light up more than the background, creating the so called “TBR” (tumor-to-background ratio). The conjugate product is lighted up using a light source creating light of a specific wave length and then detecting the light re-emitted from fluorophore using a specific filter for the specific re-emitted light. In a thought ideal situation, the cancer cells will light up immediately after injection, with sufficient high relative light intensity, without any background light, and the created desired TBR on at least 1.5 would last several hours. In a practical world it is acceptable if the desired TBR on 1.5 is reached 4,500 minutes, preferably 1,200 minutes, more preferably 600 minutes, even more preferably 300 minutes, after injection and lasts at least 30 minutes. A further explanation with reference to this aspect and others are given below in relation to the present invention.

The conjugate product according to the present invention exhibits several features including some linked to its pharmacokinetic and receptor binding affinity. The receptor-targeting conjugate provides a specific combination of plasma half-life and receptor binding affinity.

In line with the above, according to one specific embodiment of the present invention the speed of which the protein (P)-ligand (L) complex takes place may be defined as

$$P + L {{⇌}^{{K}_{on}}}_{{K}_{off}} P \cdot L ,$$

where Kₒₙ is a constant of the binding reaction and where K_{off} is a constant for the dissociation of the protein-ligand complex, and wherein Kₒₙ>1×10³ M⁻¹s⁻¹ and/or K_{off}<1×10⁻¹ s⁻¹.

Moreover, according to yet another specific embodiment of the present invention, receptor binding affinity defined as IC₅₀, which is a measurement of the ligand/receptor binding affinity, on 320 nM (nano molar, 10⁻⁹ mol/L) or less using the below in vitro test method.

These aspects are further discussed below.

Below, several important aspects and features are further explained in relation to the present invention. As a start, the conjugate composition should be administered in a sufficiently high dose, being the number of molecules (mol) of the conjugate product, distributes in the distribution volume in the body, administered as systemic administration and targeted a specific receptor present on a number of cells it can bind to. This to ensure the conjugate binding to the target receptor quickly allowing a fast creation of the TBR in combination with a short plasma half-life and lasting for sufficiently long time to be useful. In short a high receptor binding affinity in combination with the short plasma-half-life creating a fast, high and long lasting TBR.

The TBR may be calculated from the relative intensity of light from tumor and background. The measurement of the light intensity may e.g. be performed using a simple commercially available camera with physical filters, such as, but not limited to, the clinically approved NIR-camera system, e.g. Fluobeam®800 (Fluoptics, Grenoble, France) or EleVision™ (Medtronic, USA). Post image recording optimization of the image using software may be applied to enhance the TBR.

A high concentration will push the equilibrium towards more conjugate being bound to the target receptor. The concentration should however not be too high as this increases the risk of toxic effects for the patient, and the increases cost for the administration beyond what is practically acceptable. According to the present invention, the administration is done systemically, preferably intravenously why the plasma concentration quickly reaches its highest concentration. The plasma concentration will decrease thereafter as the conjugate product is metabolized, exceeded (by liver), eliminated (by kidney) or distributes to distribution compartment differently to the blood. According to the present invention, the concentration shall be sufficiently high and be maintained for a sufficiently long period for the conjugate product to reach a high enough and prolonged enough concentration in the compartment relevant for the receptor targeted (e.g. plasma, tissue stoma, cerebrospinal fluid, urine) for the conjugate product to bind to the receptors. According to one specific embodiment of the present invention, the dosing is performed in the range of 0.01-1 mg product per kg human or animal (“mg/kg”). Different doses can produce the different or the same TBR depending on the properties of the conjugate product.

Furthermore, another important feature is the binding affinity to the target receptor, including the onset and the off-set. This will mark the target tumor cells quickest possible, with the highest light intensity from the marked tissue, for the longest possible time. As mentioned, the binding affinity needs to be sufficiently high and quick, and last sufficiently long time to be useful for the conjugate product's clinical application. As mentioned, the receptor binding affinity should be reached fast, defined as within 4,500 minutes, preferably 1,200 minutes, more preferably 600 minutes, even more preferably 300 minutes. Therefore, according to one specific embodiment of the present invention, the receptor binding affinity is reached within a time of 300 min, measured in vitro. Preferably, this desired binding affinity is reached already within 180 minutes, or 150 minutes, or 60 minutes, or as fast as within 30 minutes, some times already within 15 minutes or even within 5 minutes, after administration of the conjugate product. According to one specific embodiment, receptor binding affinity is measured in vitro using a receptor affinity assay. Furthermore, the desired receptor binding occupancy measured in vitro using the assay, defined as “uPAR receptor affinity assay”, should be at least 5%, preferably at least 25%, more preferred at least 50%, and in strong cases even at least 75%. Moreover, the receptor binding should according to the present invention last at least 30 minutes after that the desired receptor binding is obtained, preferably at least 60 minutes, more preferred at least 2 h, and even more preferred at least up to 48 h, measured in vivo and defined as the time the TBR is above 1.5. Furthermore, according to yet another specific embodiment, a receptor binding shall last at least 120 minutes, preferably at least 300 minutes, after administration into the human or animal body, measured in vivo as an TBR above 1.5.

In relation to the above it may be mentioned that TBR is a feature measured in vivo and is created by a combination of several other features, such as plasma half-life, but where the receptor binding affinity is one important feature. The receptor binding affinity, however, is measured in vitro using an assay as disclosed above.

Yet another important aspect is the clearance of the conjugate from plasma. This will remove background light and thereby increase the contrast (TBR). The faster the conjugate is removed from blood plasma after the receptor binding the better. As mentioned above, the conjugate should be removed from plasma to make the conjugate bound to the receptor visible for the surgeon to distinguish tissue where the product is bound and tissue where it is not bound. This may be measured as plasma half-life which is the time it takes for the concentration in plasma to be reduced with 50%. According to the present invention, the plasma half-life has a maximum of 4,500 minutes, preferably 1,200 minutes, more preferably 600 minutes, even more preferably 300 minutes. As said, the combination of the receptor affinity and the plasma half-life are property feature results in the TBR (tumor-to-background ratio). According to the present invention said TBR is at least 1.5 and reaches that level within 300 minutes after administration into the human or animal body and stays above 1.5 at least 30 minutes after that this level has been obtained. Preferably, said TBR reaches at least 2, such as at least 3, 4, 5, 7, 9 or even at least 10. Moreover, said TBR level of at least 1.5 needs to reach within at least 60 minutes, preferably within 30 minutes, such as even within 15 minutes. This further implies that the conjugate according to the present invention has a half-life of maximum 4,500 minutes, preferably 1,200 minutes, more preferably 600 minutes, more preferably 300 minutes, preferably maximum 180 minutes, more preferably maximum 120 minutes, even more preferred maximum 60 minutes, or most preferable less than 30 minutes. Furthermore, this can also be measured in conjugate product removed from plasma per hour such as at least 5%/hour, preferably at least 10%/hour, more preferred at least 20%/hour, or even higher, such as at least 50%/hour.

Moreover, also sensitivity for cancer is of interest in relation to the present invention. According to one specific embodiment of the present invention, the receptor-targeting conjugate has a sensitivity for detection of cancer tissue of at least 60%, preferably above 70%, more preferably above 80% and most preferably above 90%. Due to some or all of its properties. With sensitivity for detection of cancer tissue is understood the probability of a positive test result of the tissue sample (light up) when the tissue sample contains disease evaluated microscopic judged by a pathologist. An example is that the surgeon removed 10 tissues samples that he/she believes is cancer and seven of them is confirmed histologically is cancer given a selectivity of 7/10 (70%). If 100% of the tissue samples removed by the surgeon believing is cancer are proven to be cancer, the sensitivity is 100%.

One other aspect in relation to the aspects above are the place of excretion and elimination. Different conjugate types according to the present invention excretes and/or eliminates in different organs and are as such not suitable for cancer types localized in these organs. Moreover, the type of indication and target receptor are also important according to the present invention. The preference here is that the receptor needs to be expressed on the cancer the patient has to the maximal benefit for the operator (e.g. the surgeon). Furthermore, it is of specific interest that the receptor is expressed on the right part of the cancer. This is of interest as normally the middle of the cancer is easy to see and remove by the surgeon. The boarders and local invasive outgrowths from the cancer, however, are more difficult to see and separate from normal tissue by the surgeon, hence more difficult for the surgeon to remove and/or to save normal tissue.

Ideally the conjugate product is administered to the patient when the surgeon undertakes the surgical procedure of removing the cancer, including when the surgeon investigates the completeness of the surgical procedure by investigating the removed cancer tissue and by investigating if there are any cancer cells left in the patient right after having removed the cancer tissue, investigate the tissue removed or when planning the post-surgery treatment. This is e.g. right before or under the surgery, or right after, during or before the anesthesia. This is a huge improvement in comparison with other known alternatives which must be administered 6 hours, or even 1-2 day in advance of surgery to be ready for use during surgery and not even in every case produce a satisfactory TBR or having a satisfactory sensitivity or specificity. The combination of the features of receptor binding and plasma clearance according to the present invention will allow such improved use. In other words, the conjugate product according to the present invention has a pharmacokinetic profile that allows administration as near before the time of use for the surgeons as possible, e.g. around anesthesia. It may furthermore be said that the conjugate product according to the present invention enables that the time from administration to first feasible time for use is within at least 2,000 minutes, 300 minutes, or 120 minutes, such as preferably within 60 minutes, or even within 30 minutes, e.g. within 15 minutes from administration.

As is evident from above, the receptor-targeting conjugate comprises a fluorophore; a molecule binding to the receptor; and a linker group which covalently links the fluorophore to the molecule binding to the receptor. The fluorophore may be of different type according to the present invention. According to one embodiment of the present invention the fluorophore is a near-infrared I fluorophore or a near-infrared II fluorophore. Interesting examples are NIR-I fluorophore selected from the group consisting of ICG, Methylene blue, 5-ALA, Protoporphyrin IX, IRDye800CW, ZW800-1, Cy5, Cy7, Cy5.5, Cy7.5, IRDye700DX, Alexa fluor 488, Fluorescein isothiocyanate. According to another specific embodiment of the present invention the fluorophore may be a NIR-II fluorophore selected from the group consisting of Flav7, CH1055, Q1, Q4, H1, IR-FEP, IR-BBEP, IR-E1, IR-FGP, IR-FTAP.

Moreover, the peptide may also of different type. According to one specific embodiment of the present invention, the peptide is chosen from the group consisting of:

-Asp-Cha-Phe-ser-arg-Tyr-Leu-Trp-Ser; and

-Asp-Cha-Phe-ser-arg-Tyr-Leu-Trp-Ser-NH₂.

Furthermore, the amino acid may be selected from proteinogenic amino acids and non-proteinogenic amino acids, which includes natural amino acids and synthetic amino acids. In relation to this, it may further be mentioned that the natural amino acids may include C-alpha alkylated amino acids such aminoisobutyric acid (Aib), N-alkylated amino acids such as sarcosine, and naturally occurring beta-amino acids such as beta-alanine. Further, the synthetic amino acids may include amino acids with non-proteinogenic side-chains such as cyclohexyl alanine, gamma-amino acids, and dipeptide mimics. The term dipeptide mimics may be interpreted as an organic molecule that mimics a dipeptide by displaying the two amino acid side-chains, e.g., having a reduced amide bond linking two residues together. Amino acids with non-proteinogenic side-chains may also include amino acids with side-chains with restricted motion in chi-space. The term restricted motion in chi-space may be interpreted as restricted flexibility in the rotation of the side-chain groups. The oligopeptides may consist of up to fifty amino acids and may include dipeptides, tripeptides, tetrapeptides, and pentapeptides, and may further be made up by proteinogenic amino acids and non-proteinogenic amino acids.

Furthermore, the present invention also refers to a pharmaceutical composition comprising the receptor-targeting conjugate according to the present invention together with at least one pharmaceutically acceptable carrier or excipient.

As is evident from above, the conjugate according to the present invention is intended to be used in cancer surgery, cancer therapy and/or cancer diagnosis. In line with this, according to one embodiment of the present invention, the receptor-targeting conjugate according to the present invention is provided for use in cancer surgery, cancer patient risk stratification, cancer therapy or diagnosis, such as for use in optical imaging/-fluorescence imaging (FLI) of cancer. It should be said that the conjugate according to the present invention finds use in several different types of indications. Some examples are glioblastoma, glioma, primary or secondary lung cancer, colorectum cancer, breast cancer, prostate cancer, stomach cancer, gastric cancer, primary or secondary liver cancer, thyroid cancer, bladder cancer, esophagus cancer, pancreas cancer, kidney cancer, corpus uteri cancer, cervix uteri cancer, melanoma, brain (incl. central and peripheral nervous system and supporting tissue) cancer, ovary cancer, gallbladder cancer, head and neck (e.g. lip, oral cavity, larynx, nasopharynx, oropharynx, hypopharynx) cancer, multiple myeloma, testis cancer, vulva cancer, salivary glands cancer, mesothelioma cancer, penis cancer, Kaposi sarcoma, vagina cancer, neuroendocrine tumors, neuroendocrine carcinomas.

In imaging there is of course also equipment present. The conjugate product according to the present invention contains a fluorescent chemical element that can re-emit light upon light excitation. The excitation and emitted light are specific to the fluorophore used. The excitation light can come from a laser such as e.g. with a wavelength between 600 nm (nano meter) and 900 nm. The emitted light from the fluorophore is typically detected by a camera using a mechanical or software-based filter e.g. detecting light between 750 nm and 950 nm. The equipment used may be a surgical robot, surgical microscope, endoscope or a handheld device. The specification of the light source (e.g. the laser) and light detector (e.g. camera with filter) depends on the fluorophore chosen.

Several different types of procedures may be used according to the present invention. Non-limiting examples of surgical procedures are, day care surgery, open surgery, minimal invasive surgery and robot assisted surgery.

It can also be surgeries with different purpose. Non-limiting examples of surgical purposes are: Curative surgery (aims to remove all the cancerous tumor from the body—this is included into the marked size calculation), Preventive surgery (is used to remove tissue that does not contain cancerous cells but may develop into a malignant tumor, e.g. a polyps in the colon), Diagnostic surgery (helps to determine whether cells are cancerous, e.g. taking a biopsy with the aim of making a diagnostic or screenings test, such e.g. looking for colon rectal malignant polyps using a colorectal scope), Staging surgery (works to uncover the extent of cancer e.g. laparoscopy (a viewing tube with a lens or camera is inserted through a small incision to examine the inside of the body)), Debulking surgery (removes a portion, though not all, of a cancerous tumor. It is used in certain situations when removing an entire tumor may cause damage to an organ or the body), Palliative or supportive surgery (is used to treat cancer at advanced stages. It does not work to cure cancer, but to relieve discomfort or to correct other problems cancer or cancer treatment may have created. An example of supportive surgery is the insertion of a catheter to help with chemotherapy), Restorative surgery (is sometimes used as a follow-up to curative or other surgeries to change or restore a person's appearance or the function of a body part. E.g. following women with breast cancer), or Corrective surgery (is a reoperation to solve problems after surgery (or other treatments), e.g. bleedings or infection).

The present invention is also directed to a method intended for cancer therapy, staging or diagnosis, such as in optical imaging/fluorescence imaging (FLI) of cancer. Therefore, according to one specific embodiment of the present invention there is disclosed a method comprising providing the receptor-targeting conjugate according to the present invention, said method also comprising
binding of the fluorophore to the receptor with a receptor binding occupancy of at least 5%, measured in vitro using a receptor affinity assay, and a maximum of the receptor binding within 4,500 minutes, preferably 1,200 minutes, more preferably 600 minutes, even more preferably 300 minutes, after administration into the human or animal body, resulting in the TBR of at least 1.5 is reached within 4,500 minutes, preferably 1,200 minutes, more preferably 600 minutes, even more preferably 300 minutes, after administration into the human or animal body; and
using the receptor-targeting conjugate in cancer therapy or diagnosis, such as in optical imaging/fluorescence imaging (FLI) of cancer.

The method may be directed to a method which involves to diagnose an anatomical structure, guide the surgeon/robot, assist the surgeon/robot, increase survival, increase the number of cancer tissues removed under surgery, increase the amount of cancer tissue removed under surgery, increase the quality of life, reduce the amount of normal tissue removed, reduce the amount of life/quality/cosmetic critical normal tissue removed, increase the certainty, reduce surgery time, improve the quality of surgery, improve the quality assurance of surgery, reduce the cost of surgery, improve the surgeon's performance, and/or improve the surgical outcome in any other way.

Furthermore, the present invention is also directed to a method involving optical imaging. Therefore, according to one specific embodiment there is disclosed an optical imaging method comprising the steps of:

(i) administering of a receptor-targeting conjugate according to the present invention accumulating in a target tissue,
(ii) allowing time for the receptor-targeting conjugate to accumulate in the target tissue and establishing a receptor binding within 4,500 minutes, preferably 1,200 minutes, more preferably 600 minutes, even more preferably 300 minutes, after administration into the human or animal body, and with a TBR of at least 1.5 which TBR level is reached within 4,500 minutes, preferably 1,200 minutes, more preferably 600 minutes, even more preferably 300 minutes, after administration into the human or animal body;
(iii) illuminating the target tissue with light of a wavelength absorbable by the fluorophore; and
(iv) detecting fluorescence emitted by the fluorophore and forming an optical image of the target tissue.

To summarize some different aspects of the present invention, the following may be stated. The conjugate according to the present invention exhibits the following features:

A large TBR is generated fast and lasting for a long time (during surgery) which is reached as a combination of:
Is obtained at a sufficient high concentration in the plasma (molar measure in the SI unit mol/m³);
The sufficiently high concentration in the blood is translated into a sufficiently high concentration in close proximity to the receptors on the cancer cells wished to be identified
Binds with high affinity—fast with a high affinity and lasting for a long time to the target receptor;
Is removed from plasma quickly;
Is needed in totally acceptable administration doses with no toxicity causing adverse effects and thereby being acceptable by patient, and with acceptable costs to the payer of the health care in which the conjugate is a part of;
Is highly specificity to specific receptor(s) that is extensively expressed on the cancer of interest, in the part of the cancer of interest and with a high selectivity to the cancer compared to normal tissue in near proximity to the cancer; and
Is detectable with available/existing equipment;

The given features above may be measured and analyzed by different means and equipment.

### Expressions, Data and Measurement According to the Present Invention

First there is provided some background information.

In relation to the present invention the expression “systemic administration” should be seen as a route of administration of a compound or substance into the blood circulatory system or into its close proximity so that the entire body is affected. Administration can take place via enteral administration (absorption of the drug through the gastrointestinal tract) or parenteral administration (generally injection, infusion, or implantation). This is in contrast to topical administration where the administration e.g. is done through the mouth (orally) or on the skin (topically).

Moreover, a “linker group” is a molecule that form the interaction between two other molecules and where their respective functions to a large degree is maintained, e.g. where a peptide binds to a receptor and a fluorophore lights up. The linker binds the two together (the peptide and the fluorophore) where their desired properties are preserved in total or partially. This is e.g. that the peptide still binds to the receptor and the fluorophore lights up.

Furthermore, the expression “ligand/receptor binding affinity” is the strength of the binding interaction between the ligand for its target, e.g. a single molecule (such as a peptide linked to a fluorophore) binding to its receptor (such as uPAR).

Regarding measurements, the expression “IC₅₀” is a measurement of the ligand/receptor binding affinity. It is the concentration where 50% of a competitive binding is achieved in equilibrium. IC₅₀ depends on assay conditions, such as concentration of the compound tested. IC₅₀ is in here measured by the inhibitory effect of 3-fold dilution series of the ligand to which the IC₅₀ is being measured on, for the uPAR/uPA as an example, the interaction measured by surface plasmon resonance (‘SPR’). A high density of prio-uPA^{S356A} is immobilized on a CM5 chip to enforce complete mass-transport-limitation (MTL) and low levels of uPAR (4 nM) was analyzed to ensure a constant association rate. % active uPAR in solution was calculated based on a standard curve recorded immediately before analyses (Gårdsvoll et al 2011 JBC 286, 33544-33556). This is also the assay defined in here as the receptor affinity assay used to determine the relative receptor binding of a conjugate product. The concentration of the conjugates is calculated as if the in vivo dose used is distributed in the extracellular part of the blood (approximately 2.5 L in a human person of 70 kg), so if the in vivo dose is 1 mg in total to the human on 70 kg, the concentration corresponds to 0.4 mg/L in the in vitro assay (or converted to mol per liter depending on the conjugates weight per mol). This assay is specifically used to determine the relative receptor binding for uPAR binding conjugate products but similar assays exists for other receptors.

The speed of which the protein-ligand complex takes place and its life span is important in relation to the present invention, but generally an overlooked aspect of ligand binding by macromolecules. The simplest binding reaction is as follows.

$$P + L {{⇌}^{{K}_{on}}}_{{K}_{off}} P \cdot L$$

Kₒₙ is the constant of the binding reaction. Its units are M⁻¹×s⁻¹. K_{off} is the constant for the dissociation of the protein-ligand complex. The dimension of K_{off} is time⁻¹. Kd is the equilibrium constant for the dissociation equilibrium, it is equal to Kₒₙ/K_{off}, and its units are M.

With reference to the present invention a certain characteristic is needed of the ligand/receptor binding. The on-binding needs to be relatively fast to secure that the time between administration of the compound and the use is not too long. The off-binding needs to be relative long as it determines for how long time the compound will light up the receptor and thereby the cancer tissue.

With reference to the present invention, IC₅₀ is a measurement of the ligand/receptor binding affinity, has a value of 320 nM or less. Furthermore, according to one specific embodiment of the present invention Kₒₙ is >1×10³M⁻¹s⁻¹ preferentially>1×10⁵ M⁻¹s⁻¹. Moreover, according to yet another specific embodiment of the present invention, K_{off} is <1×10⁻¹s⁻¹, preferentially<1×10⁻²s⁻¹.

Moreover, “plasma half-life” is the time it takes for the concentration in plasma to be reduced with 50% measured in time (seconds or minutes or hours).

1. A receptor-targeting conjugate comprising:
a fluorophore;
a molecule binding to the receptor; and
a linker group which covalently links the fluorophore to the molecule binding to the receptor,
wherein the conjugate is adapted to
be administered systemically into a human or animal body;
provide receptor binding at least within 4,500 minutes, preferably 1,200 minutes, more preferably 600 minutes, even more preferably 300 minutes;
be removed from plasma to make the conjugate bound to the receptor visible, measured as plasma half-life which is the time it takes for the concentration in plasma to be reduced with 50%, and wherein the plasma half-life has a maximum of 4,500 minutes, preferably 1,200 minutes, more preferably 600 minutes, even more preferably 300 minutes;
provide receptor binding lasting at least 30 minutes;
and wherein the receptor binding affinity, the time it takes to reach the desired receptor binding, the lasting of the receptor binding and plasma half-life translate into a TBR (tumor-to-background ratio) of at least 1.5 and reaches that level within 4,500 minutes, preferably 1,200 minutes, more preferably 600 minutes, even more preferably 300 minutes, after administration into the human or animal body and stays above 1.5 at least 30 minutes after that this level has been obtained.

2. The receptor-targeting conjugate according to claim 1, wherein the conjugate is a peptide conjugate and wherein the peptide conjugate comprises a peptide binding to the receptor.

3. The receptor-targeting conjugate according to claim 1, wherein the targeting receptor is urokinase Plasminogen Activator Receptor (uPAR), tissue factor (TF), epidermal growth factor receptor (EGFR), prostate-specific membrane antigen (PSMA), Vascular Endothelial Growth Factor (VEGF), Folate receptor, matrix metalloproteinase-2 (MMP-2), membrane type-I MMP, transmembrane inhibitor of metalloproteinase-2 (TIMP2), CIC-3 chloride ion channels, disaccharides and other glycans or a glyco-phosphatidylinositol (GPI)-anchored cell membrane receptors.

4. The receptor-targeting conjugate according to claim 1, wherein the speed of which the protein (P)-ligand (L) complex takes place may be defined as
$$P + L {{⇌}^{{K}_{on}}}_{{K}_{off}} P \cdot L$$
where Kₒₙ is a constant of the binding reaction and where K_{off} is a constant for the dissociation of the protein-ligand complex, and wherein Kₒₙ>1×10³ M⁻¹s⁻¹ and/or K_{off}<1×10⁻¹ s⁻¹.

5. The receptor-targeting conjugate according to claim 1, wherein receptor affinity is measured as IC₅₀, which is a measurement of the ligand/receptor binding affinity, on 320 nM or less.

6. The receptor-targeting conjugate according to claim 1, wherein the receptor binding affinity is reached within a time of 300 min measured in vitro.

7. The receptor-targeting conjugate according to claim 1, wherein a receptor binding lasts at least 120 minutes, preferably at least 300 minutes, after administration into the human or animal body measured using a TBR above 1.5.

8. The receptor-targeting conjugate according to claim 1, wherein the receptor-targeting conjugate has a sensitivity for detection of cancer tissue of at least 60%, preferably above 70%, more preferably above 80% and most preferably above 90%.

9. The receptor-targeting conjugate according to claim 1, wherein receptor binding affinity is measured in vitro using a receptor affinity assay.

10. The receptor-targeting conjugate according to claim 1, wherein the receptor binding occupancy reaches at least 5%, preferably 25%, more preferably at least 50% measured in vitro using a receptor affinity assay.

11. The receptor-targeting conjugate according to claim 1, wherein the fluorophore is a near-infrared I fluorophore or a near-infrared II fluorophore.

12. The receptor-targeting conjugate according to claim 1, wherein the fluorophore is IRDye800CW or indocyanin green (ICG).

13. The receptor-targeting conjugate according to claim 1, wherein the peptide is chosen from the group consisting of:
-Asp-Cha-Phe-ser-arg-Tyr-Leu-Trp-Ser; and
-Asp-C ha-Phe-ser-arg-Tyr-Leu-Trp-Ser-N H2.

14. The receptor-targeting conjugate according to claim 1, for use in cancer therapy or diagnosis, such as for use in optical imaging/-fluorescence imaging (FLI) of cancer.

15. A pharmaceutical composition comprising the receptor-targeting conjugate according to claim 1 together with at least one pharmaceutically acceptable carrier or excipient.

16. A method comprising providing the receptor-targeting conjugate according to claim 1, said method also comprising
binding of the fluorophore to the receptor with a receptor binding occupancy of at least 5%, measured in vitro using a receptor affinity assay, and a maximum of the receptor binding within 4,500 minutes, preferably 1,200 minutes, more preferably 600 minutes, even more preferably 300 minutes, after administration into the human or animal body, resulting in the TBR of at least 1.5 is reached within 4,500 minutes, preferably 1,200 minutes, more preferably 600 minutes, even more preferably 300 minutes, after administration into the human or animal body; and
using the receptor-targeting conjugate in cancer therapy or diagnosis, such as in optical imaging/fluorescence imaging (FLI) of cancer.

17. An optical imaging method comprising the steps of:
(i) administering of a receptor-targeting conjugate according to claim 1 accumulating in a target tissue,
(ii) allowing time for the receptor-targeting conjugate to accumulate in the target tissue and establishing a receptor binding within 4,500 minutes, preferably 1,200 minutes, more preferably 600 minutes, even more preferably 300 minutes, after administration into the human or animal body, and with a TBR of at least 1.5 which TBR level is reached within 4,500 minutes, preferably 1,200 minutes, more preferably 600 minutes, even more preferably 300 minutes, after administration into the human or animal body;
(iii) illuminating the target tissue with light of a wavelength absorbable by the fluorophore; and
(iv) detecting fluorescence emitted by the fluorophore and forming an optical image of the target tissue.
